(19) United States
(12) Patent Application Publication
(10) Pub. No.: US 2022/0057399 A1
(43) Pub. Date: Feb. 24, 2022

(54) SAMPLE TREATMENT SOLUTION, METHOD FOR REMOVING CONTAMINANT PROTEINS AND DETECTION METHOD THEREFOR

(71) Applicant: Canon Medical Systems Corporation, Otawara-shi (JP)

(72) Inventors: Seiko YOSHIMURA, Nasushiobara (JP); Satoshi YAMANE, Otawara (JP)

(73) Assignee: Canon Medical Systems Corporation, Otawara-shi (JP)

(21) Appl. No.: 17/445,410

(22) Filed: Aug. 19, 2021

(30) Foreign Application Priority Data
Aug. 21, 2020 (JP) 2020-140063

(51) Int. Cl. G01N 33/569; G01N 33/543

(52) U.S. Cl. CPC G01N 33/56983; G01N 2333/11; G01N 2446/80; G01N 33/54326

(57) ABSTRACT

According to one embodiment, a sample treatment solution for detecting a detection target contained in a sample, includes a carrier supporting an active ester group.

### CROSS-REFERENCE TO RELATED APPLICATIONS

This application is based upon and claims the benefit of priority from Japanese Patent Application No. 2020-140063, filed Aug. 21, 2020, the entire contents of which are incorporated herein by reference.

### FIELD

Embodiments described herein relate generally to a sample treatment solution, a method for removing contaminant protein, and a detection method therefor

### BACKGROUND

A method utilizing an antigen-antibody reaction is employed to detect antigens such as viruses and bacteria contained in samples. Here, due to the effect of foreign substances contained in the sample, there is a risk of false positives, in which the sample is judged positive even when it does not contain a target to be detected. In order to reduce this effect, a blocking agent which inhibit the adsorption of foreign substances to antibodies and absorbents which pre-adsorb foreign substances are added.

However, with the above-described techniques, it has not been possible to effectively remove contaminant proteins (such as immunoglobulins, fibrin and hematopoietic cells) contained in samples originated from biological materials. One of the objects of the embodiments disclosed herein and in the drawings is to provide a sample treatment solution with which false positives can be suppressed and a target can be accurately detected, a method for removing contaminant proteins, and a detection method therefore. But, the object of the embodiments disclosed in this specification and the drawings is not limited to the above-mentioned one. Objects corresponding to the effects of the structures discussed in the embodiments described below may be considered as other objects.

### BRIEF DESCRIPTION OF THE DRAWINGS

FIG. 1 is a flowchart showing an example of a detection method according to an embodiment.

FIG. 2 is a schematic diagram showing an example of formation of an active ester group according to the embodiment.

FIG. 3 is a schematic diagram showing an example of a carrier which carries a sample and an active ester group according to the embodiment.

FIG. 4 is a flowchart showing an example of a detection method according to an embodiment.

FIG. 5 is a cross-sectional view of an example of a detection device used in the detection method of the embodiment.

FIG. 6 is a graph showing experimental results of Example 1 in optical attenuation (%) of each mixture.

FIG. 7 is a graph showing experimental results of Example 2 in optical attenuation (%) of each mixture.

### DETAILED DESCRIPTION

In general, according to one embodiment, a sample treatment solution for detecting a target contained in a sample, comprises a carrier supporting an active ester group.

Embodiments of a sample treatment solution, a method for removing contaminant proteins and a detection method therefore will be described hereinafter with reference to the accompanying drawings.

(Method for Removing Contaminant Proteins)

A method for removing contaminant proteins from a sample will now be described.

In this specification, the term “removing” means not only physically removing a contaminant protein from a sample, but also separating, when a target to be detected (hereinafter, referred to as a “detection target”) is in a sample, a contaminant protein from the detection target in the sample.

The sample is, for example, a biological sample. The biological sample is, for example, of animal origin. The biological sample of animal origin should preferably be a material containing a mucosal component (for example, mucin), such as nasal mucosa, nasal wipe, nasal discharge, nasal aspirate, nasal wash, pharyngeal wipe, saliva, pharyngeal mucosa, oral mucosa, oral wash, or sputum.

The animals are, for example, mammals, humans, other primates, cattle, sheep, goats, horses, dogs, cats, rabbits, rats, mice, birds, fish and the like. The animals should preferably be humans.

The samples may be other biological materials such as blood, serum, lymph fluid, spinal fluid, tears, breast milk, amniotic fluid, semen, urine, stool, sweat, cells, tissues, biopsies, cultured cells, cultured supernatants, cell extracts, or mixtures in any combinations thereof, environmental materials such as soil, river water, sea water, ground water, water and sewage water, plant-originated materials, food or beverage-originated materials, or any combinations thereof.

The target of detection may be, for example, a microorganism such as a virus (for example, influenza virus), a bacterium (for example, hemolytic streptococcus), or a fungus, or any combination thereof. Alternatively, the detection target may be other substances, such as nucleic acids, proteins, etc., according to the objective of detection.

The method for removing contaminant proteins comprises a contacting step (S1) of bringing a carrier supporting an active ester group into contact with a sample as shown in FIG. 1.

The contaminant proteins are proteins which may be contained in a sample and are not originated from the target of detection, such as immunoglobulins, fibrin, or blood cells. If a contaminant protein is present in the sample, the detection thereof may cause a false positive.

The carrier which supports the active ester group is, for example, magnetic beads, Sepharose beads, polystyrene beads, or microspheres.

In this specification, the term “support” means that the carrier contains an active ester group fixed to its surface by any method. For example, the active ester group is bonded to the surface of the carrier either directly or via a linker. The linker may be, for example, an atom or a molecule. The linker is bonded to the carrier, for example, by chemical, electrical, or physical bonds. For example, a polymer surface, that is, the surface of a carrier coated with a polymer may be modified to introduce an active ester group, to be used.

The active ester group can be obtained, for example, by activating a carboxy group with a carbodiimide. Such an active ester group should preferably be, for example, N-hydroxysuccinimide (NHS) esters, which is represented by the following chemical formula.

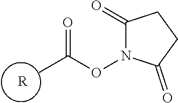

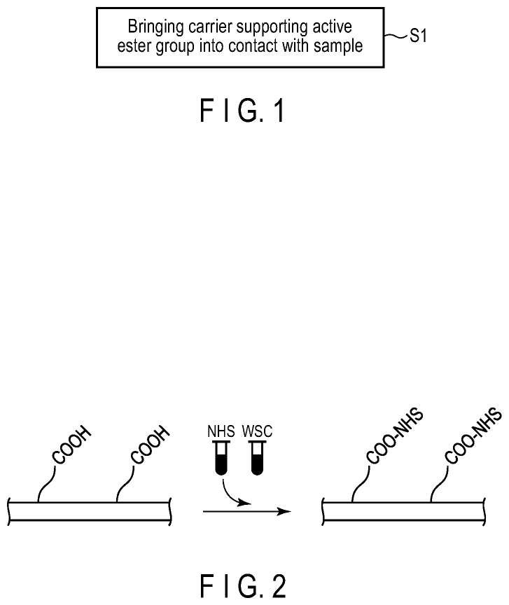

The NHS ester can be formed, for example, by activating a carboxy group with NHS and a carbodiimide as a condensing agent, as shown in FIG. 2. The carbodiimide is, for example, water soluble carbodiimide (WSC). For example, by activating a carrier supporting the carboxy group as described above, a carrier containing an NHS ester group can be obtained.

The contacting step (S1) is carried out, for example, by mixing the sample and a solution supporting the active ester group. In the mixture, the carrier supporting the active ester group contained in the solution and the sample are brought into contact with each other.

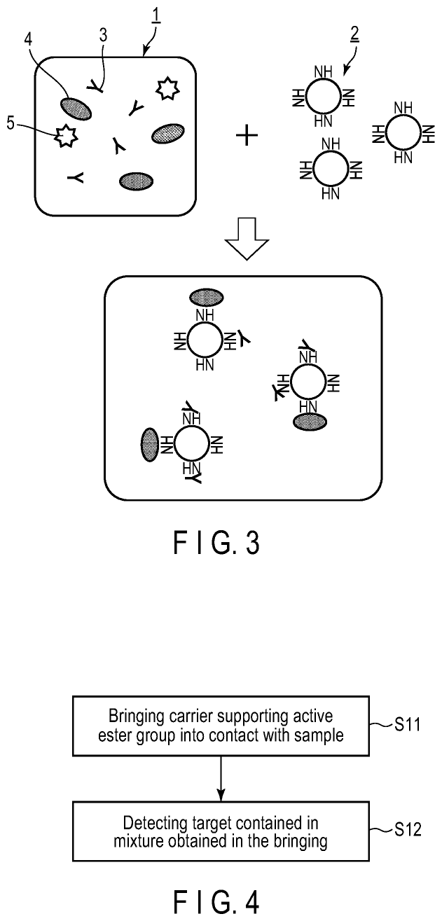

An example of the mechanism by which contaminant proteins contained in a sample are removed by a carrier supporting an active ester group by the above-described contacting step (S1) will be explained with reference to FIG. 3. As shown in FIG. 3, active ester groups supported by the carrier 2 are present on the surface of the carrier 2. In a sample 1, contaminant proteins 3 and 4 are adsorbed by the active ester groups on surfaces of carriers 2. In this adsorption process, detection targets 5 are not adsorbed on the carrier 2.

With the contacting step (S1), the detection targets and the contaminant proteins are separated from each other, and thus the contaminant protein can be removed.

Then, when the carrier 2 is, for example, magnetic beads, a magnetic field is applied to the mixture of the sample 1 and the solution containing the carrier 2 supporting the active ester group, and thus the carrier 2 which adsorbs the contaminant proteins 3 and 4 and the carrier 2 which does not adsorb the contaminant proteins 3 and 4 can be both recovered. Alternatively, the detection target 5 can be selectively recovered by capturing it with some other capture material such as an antibody after the contacting step.

The sample after the contacting step (S1) can be subjected to a detection method of the detection target which, for example, utilizes an antigen-antibody reaction. But the detection method is not limited to the antigen-antibody reaction, but can also be used for nucleic acid amplification, for example. Examples of the nucleic acid amplification methods include PCR or isothermal amplification methods.

According to the method for removing contaminant proteins of the embodiment, for example, prior to the detection utilizing an antigen-antibody reaction, contaminant proteins contained in a sample are removed, and thus the occurrence of false positives can be suppressed, making it possible to perform more accurate detection.

(Detection Method)

A method of detecting a target in a sample using a carrier supporting an active ester group will now be described. As shown in FIG. 4, the method includes a contacting step (S11) of bringing a carrier supporting an active ester group and a sample into contact with each other, and a detection step (S12) of detecting the detection target contained in the mixture obtained in the contacting step.

An example of the detection method utilizing an antigen-antibody reaction will be described.

First, a sample is collected. Here, an example in which the sample is nasal aspirate or nasal swab will be described.

The method of collecting nasal aspirate can be can be carried out, for example, as follows. First, a sufficiently sterilized suction tube is prepared and one end is connected to an aspirator and the other end to a suction catheter. The suction catheter should be sterilized in a similar manner to that for the suction tube. Next, the suction catheter is inserted to the subject's nasal cavity and the aspirator is operated to collect nasal aspirate. The collected nasal aspirate may be transferred to a container such as a microtube, test tube or beaker.

The method of collecting nasal swab can be carried out, for example, as follows. First, tissue paper is prepared. Then, the subject's nose is covered with the paper and one nostril is closed. Thereafter, the subject blows his nose slowly to apply nasal discharge onto the paper. The collected nasal discharge may be attached to a swab or cotton swab, for example.

The contacting step (S11) can be carried out in a similar manner to that described in the contacting step (S1) above, but it is preferable to carry out by mixing the sample and the sample treatment solution containing the carrier supporting the active ester group described above.

The sample treatment solution will now be described.

The sample treatment solution of the embodiment is a liquid used to treat a sample to be in a condition appropriate for detection. The term “condition” includes, for example, the pH, temperature, concentration of the sample and the presence or absence of contaminants in the sample.

The sample treatment solution further contains a solvent. Usable examples of the solvent are saline solution, PBS buffer, acetate buffer, Bis-Tris buffer, PIPES buffer, HEPES buffer and BES buffer.

The sample treatment solution should further contain a reagent such as a surfactant. The surfactant blocks non-specific antigen-antibody reactions of mucosal components and/or viscous substances, etc. contained in the sample. Such a viscous substance is, for example, mucin. Usable examples of the surfactant are a cationic surfactant, an anionic surfactant, or an amphoteric surfactant. As a cationic surfactant, for example, 0-[2-hydroxy-3-(trimethylammonio)propyl]hydroxyethyl cellulose chloride can be used.

The reagent to be contained in the sample treatment solution is not limited to the surfactant, but appropriate ones may be selected according to the type of sample used and the type of detection method. For example, a reagent for detection may be further contained.

The sample treatment solution can be obtained, for example, by adding a carrier supporting the above-described active ester group to the above-described solvent. If some other reagent such as a surfactant is to be contained, it may be further added.

By mixing the nasal aspirate or nasal swab solution with the sample treatment solution, the carrier supporting the active ester group contained in the sample treatment solution comes into contact with the nasal aspirate solution as the sample, and thus a mixture is obtained. Thus, as described in the method for removing contaminant proteins, the contaminant proteins contained in the sample can be adsorbed onto the carrier supporting the active ester group, to be removed.

After the contacting step (S11) described above, the detection target in the mixture is detected (detection step (S12)). This step is performed, for example, using a detection device. For example, when a protein which is the detection target in the mixture and the antibody specifically bind to each other by the antigen-antibody reaction, the amount of the protein in the mixture decreases. With this mechanism, as the detection device, for example, an optical sensor can be used, which can detect a change in the amount of protein in the mixture obtained in the contacting step (S11) as a change in optical signal.

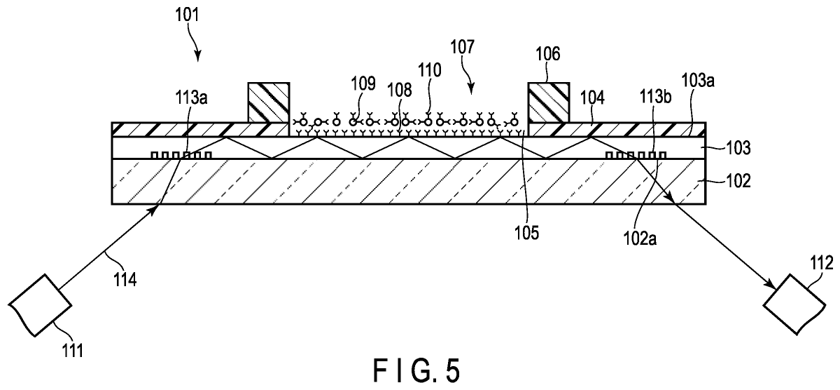

An example of the detection device will be described with reference to FIG. 5. A detection device 101 comprises a substrate 102 made of a light-transmissive material and an optical waveguide 103, which is a planar optical waveguide provided on a main surface 102a of the substrate 102 and made of a material such as a light-transmissive resin. A low-refractive resin layer 104 is provided on a main surface 103a of the optical waveguide 103. An opening 105 is formed in the low-refractive resin layer 104 so as to expose a portion of the area of the main surface 103a of the optical waveguide 103. Further, well walls 106 are provided along edges of the opening 105 in the low-refractive resin layer 104. The opening 105, the well wall 106, and the portion of the main surface 103a of the optical waveguide 103 exposed from the opening 105 define a well 107 for accommodating a mixture of a sample treatment solution containing a sample and the sample. A first substance 108 (for example, an antibody or the like) which specifically captures a detection target is fixed on the main surface 103a of the optical waveguide 103 located at the bottom of the well 107. Further, particles 109 are dispersed on the main surface 103a. A second substance 110 (for example, antibody or the like), which is different from the first substance 108 and specifically captures the detection target, is fixed to the particles 109.

The detection device 101 comprises a light source (for example, a laser diode) 111 which inject light into the optical waveguide 103, and a light-receiving element (for example, a photodiode) 112 which receives light output from the optical waveguide 103.

An incident-side grating 113a and an outgoing-side grating 113b are provided on the main surface 102a of the substrate 102 in the optical waveguide 103. The incident-side grating 113a is disposed at a position on the main surface 102a where light 114 from the light source 111 enters, and adjusts the angle of incidence of the light 114 so that the light 114 propagates in through the optical waveguide 103 by total reflection. The outgoing-side grating 113b is disposed at a position on the main surface 102a from which light 114 is emitted from the optical waveguide 103 and adjusts the angle of incidence of the light 114 so that light 114 is emitted from the optical waveguide 103 and received by the light receiving element 112. The gratings 113a and 113b are formed of, for example, titanium oxide (TiO₂), tin oxide (SnO₂), zinc oxide, lithium niobate, gallium arsenide (GaAs), indium tin oxide (ITO), polyimide or the like. The incident-side grating 113a and the outgoing-side grating 113b may be formed by providing a projection-and-recess shape on the surface of the substrate 102 or the optical waveguide 103.

Next, a method of detecting a detection target using the detection device 101 will be described. First, a mixture of the sample treatment solution and the sample, obtained in the contacting step (S11), is dropped into the well 107. Next, the light 114 from the light source 111 is injected to the optical waveguide 103 through the incident-side grating 113a. Thus, the light 114 propagates while being reflected in the optical waveguide 103. As a result, evanescent light is generated near the surface of the optical waveguide 103 exposed to the opening 105. Then, the light-receiving element 112 detects the intensity of the light 114 emitted from the outgoing-side grating 113b.

When a detection target exists in the mixture, the detection target bonds to the first substance 108 and the second substance 110, and the particles 109 are fixed to the surface of the optical waveguide 113. Thus, the particles 109 fixed on the surface of the optical waveguide 113 are involved in the absorption and scattering of evanescent light, which causes attenuation of the intensity of the evanescent light. As a result, the light intensity detected by the light-receiving element 112 decreases. When a detection target does not exist in the mixture, the particles 109 are not fixed to the surface of the optical waveguide 113, but dispersed in the mixture, and therefore the decrease in light intensity does not occur.

If the rate of decrease (attenuation rate) of the light intensity is higher than a predetermined threshold, it can be judged that the detection target is present (positive), and if it is lower than the threshold, it can be judged that the detection target is not present or, if present, its amount is very small (negative).

Conventionally, contaminant proteins contained in samples are involved in the absorption and scattering of evanescent light, and non-specific detection, in which the detected light intensity decreases, may occur even when the sample does not contain any detection target. But with use of the sample treatment solution of the embodiment, non-specific detection caused by contaminant proteins contained in the sample, which may cause false positives, is suppressed, thereby making it possible to accurately detect the detection target. In particular, when the contaminant protein is immunoglobulin, fibrin, or hematopoietic cells, the non-specific detection can be more accurately suppressed.

According to the detection method of the embodiment, the contaminant proteins contained in the sample can be removed, and the change in the amount of protein in the mixture due to the antigen-antibody reaction can be detected as a change in the optical signal. In this way, it is possible to detect the target more accurately and more rapidly.

### EXAMPLES

Examples in which the sample treatment solution of the embodiment is prepared and used to detect a type A influenza virus as a detection target, will be described. But, the embodiments of the present invention are not limited to these examples.

### Example 1: Evaluation on the Effect in the Removal of Contaminant Proteins in Nasal Samples by NHS Ester Magnetic Beads

(Preparation of Suspension, Preparation of Activated Beads, and Treatment of Sample)

A suspension containing a buffer solution and a cationic surfactant was prepared.

Activated magnetic beads were obtained by the following procedure. First, MS160 magnetic beads (JSR Life Sciences) were washed with MES buffer. Next, a 10% solution of Sulfo-NHS prepared by the MES buffer was added to the washed magnetic beads. Further, a 10% solution of WSC prepared by the MES buffer was added thereto, and the resultant was stirred for 30 seconds. After that, the beads were inverted and mixed for 1 hour at room temperature using a rotator to obtain activated magnetic beads.

Nasal discharge samples were used as samples. For nasal discharge samples, samples were collected with nasal swab paper, and nasal secretions were permeated into the swab and added to the sample treatment solution. Note that the subjects here are not infected with either influenza A or influenza B.

Next, mixtures (1) to (5) shown in Table 1 below were prepared using these material.

TABLE 1

| | Sample treatment solution | | Inactivated | Mixed solution |
|---|---|---|---|---|
| | | Activated | type A influenza | of sample |
| Mixtures | Suspension | magnetic beads | virus antigen | and PBS |
| (1) | Present | Absent | Present | Absent |
| (2) | Present | Present | Absent | Absent |
| (3) | Present | Present | Present | Absent |
| (4) | Present | Absent | Absent | Present |
| (5) | Present | Present | Absent | Present |

The method of preparation of the mixtures (1) to (5) will be described.

(1) Suspension+Inactivated Influenza a Virus Antigen

A commercially available inactivated type A influenza virus antigen was diluted by 2⁶-fold and 30 μL of the dilution was dispensed. The dispensed dilution was mixed with 400 μL of a suspension to obtain mixture (1).

(2) Suspension+Activated Magnetic Beads

400 μL of the suspension solution and 3 μL of activated magnetic beads were mixed to obtain the mixture (2).

(3) Suspension+Activated Magnetic Beads+Inactivated Influenza a Virus Antigen

A commercially available inactivated type A influenza virus antigen was diluted by 2⁶-fold and 30 μL of the dilution was dispensed. The dispensed dilution was mixed with 400 μL of the suspension and 3 μL of the activated magnetic beads to obtain mixture (3).

(4) Suspension+Sample+PBS

After the nasal discharge sample was collected, it was suspended in 500 μL of PBS to obtain a mixed solution. 100 μL of the mixed solution and 400 μL of the suspension were mixed to obtain the mixture (4).

(5) Suspension+Sample+PBS+Activated Magnetic Beads

After the nasal discharge sample was collected, it was mixed with 500 μL of PBS to obtain a mixed solution. 100 μL of the mixed solution, 400 μL of the suspension and 3 μL of activated magnetic beads were mixed to obtain the mixture (5).

(Detection)

As an immunoassay device, a detection device (product name: Rapim (registered trademark), manufactured by Canon Medical Systems Inc.) equipped with the measurement system described in FIG. 5 was used. Each of the above mixtures (1) to (5) was let stand still for a certain period of time for reaction, and then dropped respectively into the wells of the detection device. Thus, the attenuation rate of the optical signal was measured for each.

(Results)

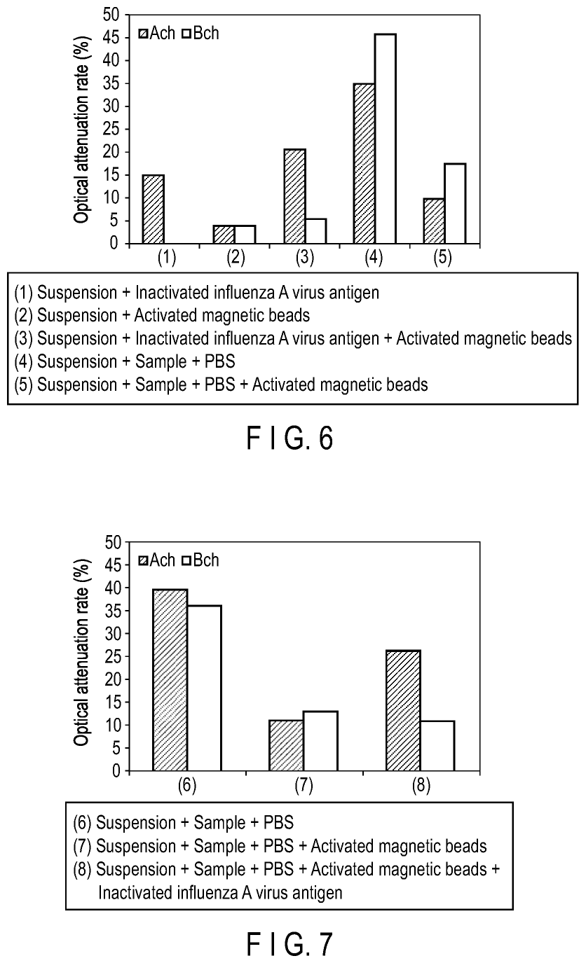

FIG. 6 shows the optical attenuation (%) of each of the mixtures. Ach is the channel used for detecting influenza A viruses and Bch is the channel used for detecting influenza B viruses.

As to the mixture (1), the optical attenuation rate was 15% for channel A and 0% for channel B, which indicates that the suspension cause no significant effect on the optical attenuation rate.

As to the mixture (2), the optical attenuation rate was about 4% in both channels A and B, which indicates there was no significant increase in the optical attenuation rate caused by the activated magnetic beads.

As to the mixture (3), the optical attenuation rate was 21% for channel A and 5% for channel B. Comparing the results of the mixtures (1) to (3) for channel A, it is clear that the optical attenuation rate is not affected if the influenza A virus antigen and the activated magnetic beads coexist.

As to the mixture (4), the optical attenuation rate was 35% for channel A and 46% for channel B. Despite the fact that the subjects were not infected with either influenza A or influenza B, a significant increase in optical attenuation was monitored as compared to the case of the mixture (1). Thus, it was indicated that the samples contained contaminant proteins that could cause a non-specific antigen-antibody reaction and result in a false positive result.

As to the mixture (5), the result was 10% for channel A and 18% for channel B. Here, as compared to the results of the mixture (4), the optical attenuation was reduced by 25% in channel A and by 18% in channel B. The difference between the mixtures (4) and (5) is the presence or absence of activated magnetic beads, and therefore it was indicated that the activated magnetic beads removed contaminant proteins and prevent non-specific antigen-antibody reactions.

From the above-provided results, it is clear that the sample treatment solution containing activated magnetic beads can remove contaminant proteins and inhibit non-specific antigen-antibody reactions.

### Example 2: Effect of Removal of Contaminant Proteins and Detection of Antigen by NHS Magnetic Beads

Using the suspension, activated magnetic beads, and samples used in Example 1, mixtures (6) to (8) listed in Table 2 below were prepared.

TABLE 2

| | Sample treatment solution | | Inactivated | Mixed solution |
|---|---|---|---|---|
| | | Activated | type A influenza | of sample |
| Mixtures | Suspension | magnetic beads | virus antigen | and PBS |
| (6) | Present | Absent | Absent | Present |
| (7) | Present | Present | Absent | Present |
| (8) | Present | Present | Present | Present |

The method of preparing the mixtures of (6) to (8) will be described.

(6) Suspension+Sample+PBS

After the nasal discharge sample was collected, it was suspended in 500 μL of PBS to obtain a mixed solution. 200 μL of the mixed solution and 400 μL of the suspension were mixed to obtain the mixture (6).

(7) Suspension+Sample+PBS+Activated Magnetic Beads

After the nasal discharge sample was collected, it was mixed with 500 μL of PBS to obtain a mixed solution. 200 μL of the mixed solution, 400 μL of the suspension and 10 μL of activated magnetic beads were mixed to obtain the mixture (7).

(8) Suspension+Sample+PBS+Activated Magnetic Beads+Inactivated Influenza a Virus Antigen

A nasal wipe sample was collected and mixed with 500 μL of PBS to obtain a mixed solution. A commercially available inactivated type A influenza virus antigen was diluted by 2⁶-fold. Then, 200 μL of the mixed solution, 400 μL of the suspension, 10 μL of activated magnetic beads and 30 μL of inactivated type A influenza virus antigen (2⁶-fold dilution) were mixed to obtain the mixture (8).

(Detection)

Each of the above-described mixtures (6) to (8) was let stand still for a certain period of time for reaction, and in each case, the mixture was dropped into the well of a detection device similar to Example 1 in respective cases, and the attenuation rate of the optical signal was measured for each case.

(Results)

FIG. 7 shows the optical attenuation (%) of each mixture. As in the case of Example 1, Ach and Bch are a channel used to detect type A influenza viruses and a channel used to detect type B influenza viruses, respectively.

As to the mixture (6), the optical attenuation rate was 40% for channel A and 36% for channel B.

As to the mixture (7), the optical attenuation rate was 11% for channel A and 13% for channel B. Similar to the results of comparison between the mixtures (4) and (5) in Example 1, it was indicated that with the addition of activated magnetic beads, contaminant proteins can be removed and the optical attenuation rate can be significantly reduced.

As to the mixture (8), the optical attenuation rate was 27% for channel A and 11% for channel B. In the channel A, the optical attenuation rate was increased by 16% as compared to the results of the mixture (7). Thus, it was demonstrated that inactivated influenza A virus antigen can be detected even in a mixture system containing a sample treatment solution, sample, PBS, and inactivated influenza A virus antigen.

These results indicate that when the carrier supporting the active ester group of the embodiment is used in detection of a target utilizing an antigen-antibody reaction, contaminant proteins can be removed and false positive judgments caused by contaminant proteins can be effectively suppressed. Therefore, it was demonstrated that when detecting a target by utilizing the antigen-antibody reaction, the detection target can be detected more accurately.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A sample treatment solution for detecting a detection target contained in a sample, comprising:
a carrier supporting an active ester group.

2. The sample treatment solution of claim 1, wherein
the active ester group is N-hydroxysuccinic acid imide (NHS) ester.

3. The sample treatment solution of claim 1, wherein
the carrier is magnetic beads or Sepharose beads.

4. The sample treatment solution of claim 1, wherein
the sample is a biological sample of human origin and is nasal aspirate or nasal swab.

5. The sample treatment solution of claim 1, wherein
the detection target is a virus and/or bacteria.

6. The sample treatment solution of claim 5, wherein
the virus is an influenza virus.

7. The sample treatment solution of claim 1, wherein
the sample further contains contaminant proteins such as fibrin, immunoglobulin, and/or blood cells.

8. The sample treatment solution of claim 1, wherein
the detection is performed utilizing an antigen-antibody reaction.

9. A method for removing a contaminant protein contained in a sample, comprising:
bringing a carrier supporting an active ester group into contact with the sample.

10. A method for detecting a target contained in a sample, comprising:
bringing a carrier supporting an active ester group into contact with the sample; and
detecting the target contained in a mixture obtained in the bringing.

11. The method of claim 10, wherein
the detecting is performed by an optical sensor which detects a change in the amount of protein contained in the mixture as a change in an optical signal, caused by an antigen-antibody reaction.
